(19) United States
(12) Patent Application Publication
(10) Pub. No.: US 2018/0078482 A1
(43) Pub. Date: Mar. 22, 2018

(54) COMPOSITION CONTAINING THEASAPOGENOL DERIVATIVE AS ACTIVE INGREDIENT

(71) Applicant: AMOREPACIFIC CORPORATION, Seoul (KR)

(72) Inventors: Yong Deog HONG, Yongin-si, Gyeonggi-do (KR); Jae Young KO, Yongin-si, Gyeonggi-do (KR); Jun Seong PARK, Yongin-si, Gyeonggi-do (KR)

(73) Assignee: AMOREPACIFIC CORPORATION, Seoul (KR)

(21) Appl. No.: 15/561,560

(22) PCT Filed: Mar. 25, 2016
(86) PCT No.: PCT/KR2016/003038

§ 371 Date: Sep. 26, 2017

(30) Foreign Application Priority Data
Mar. 31, 2015 (KR) 10-2015-0045133

(51) Int. Cl. A61K 8/63; A61K 31/56; A61Q 19/08; A61Q 5/02; A61Q 19/10; A61K 36/82; A61K 8/9789; A23L 33/105; A23L 2/52

(52) U.S. Cl. CPC A61K 8/63; A61K 31/56; A61Q 19/08; A61Q 5/02; A23V 2002/00; A61K 36/82; A61K 8/9789; A23L 33/105; A23L 2/52; A61Q 19/10

(57) ABSTRACT

The present specification discloses a composition for shrinking pores, inhibiting or improving wrinkles, promoting skin elasticity, regulating sebum, and preventing or improving acne skin, the composition containing, as an active ingredient, theasapogenol derivative 21-O-angeloyltheasapogenol E3 as one embodiment.

### CROSS-REFERENCE TO RELATED APPLICATION

This application claims the priority of Korean Patent Application No. 10-2015-0045133, filed on Mar. 31, 2015, and all the benefits accruing therefrom under 35 U.S.C. § 119, the contents of which in its entirety are herein incorporated by reference.

### TECHNICAL FIELD

The present specification discloses compositions for tightening of pores, inhibition or improvement of wrinkles, enhancement of skin elasticity, control of sebum, and prevention or improvement of acne skin, which contain a theasapogenol derivative as an active ingredient.

### BACKGROUND ART

The skin is classified into various types such as a dry type, a neutral type, an oily type, and a complex type depending on the condition of the skin surface, and this is determined by the amounts of NMF (natural moisturizing factor) and sebum. The skin maintains the moisturizing and soft condition when the amounts of NMF and sebum are well balanced, but it is likely to be oily skin or acne skin when sebum is secreted too much. Oily skin has pores on the skin surface, so that pores are present with a large amount of sebum being secreted on the skin surface, the face looks dirty, and makeup on the face is easily removed.

Hence, it is required to develop a substance which overcomes such problems and thus has a function of relieving the skin unpleasantness caused by sebum secretion, refreshing the skin, and decreasing skin irritation.

The dermis is a part composed of a connective tissue below the epidermis and filled with a macromolecular network called extracellular matrix. The part constituting the extracellular matrix is a fibrous protein composed of a polysaccharide called glucosaminoglycan or mucopolysaccharide, collagen, and elastin. Among these, collagen is the main protein constituting the extracellular matrix and acts to maintain the morphology of the tissue, and dermis thus has a great influence on the elasticity and tensile strength of the skin. Hence, when collagen is damaged due to external or internal influences, wrinkles are formed or the skin elasticity decreases and the skin is slackened. This is also a reason for that the cosmetic products which improve wrinkles have also recently used components which increase collagen synthesis such as retinol.

In addition, an increase in the size of pores is a phenomenon that the matrix structure of the dermis is relaxed and the skin around the pores is slackened. The dermis contains blood vessels, nerves, hair, biceps (also called asymmetric roots), sweat glands, and sebaceous glands. The sebaceous glands constantly produce sebum and secrete sebum from the excretory drift of the sebaceous gland to the skin surface through the pores. At this time, hyperkeratinization (phenomenon that the keratinization is not normally performed and the detachment is delayed) is likely to be caused in the pores, the thickened stratum corneum is peeled off in the hair follicles, clogs the pores, causes sebum to remain in the hair follicles, thus causes rashes and inflammation, and forms granuloma to leaves traces.

Hence, in order to effectively tighten the enlarged pores, it is required to remove the keratins on the pores thickened by an abnormal keratinization process to activate the cellular metabolism, at the same time to promote the collagen production at the dermal tissue, and thus to restore the structure of the depressed or enlarged pores.

Meanwhile, 5α-reductase is present in male hormone-reactive tissues such as sebaceous glands, hair follicles, prostate, epididymis, and is an enzyme involved in the reduction of testosterone, one of the male hormones, to dihydrotestosterone (DHT), and NADPH is required in this conversion. Testosterone is involved in male sexual dysfunction, skeletal muscle increase, male external genitalia, scrotum growth, and spermatogenesis, and dihydrotestosterone is involved in acne, increased sebum, and enlargement of the prostate in the corresponding tissues (Sugimoto et al; J.I.D. Vol. 104, No. 5, 775-778, 1995. Bruchovsky, N. et al., J.B.C. 243, 2012-2021, 1968).

Hence, in order to inhibit the side effects due to dihydrotestosterone produced by 5α-reductase, studies are underway to develop acne remedies and sebum secretion inhibitors by using inhibitors of 5α-reductase enzymes.

The above information disclosed in the Background section is only for enhancement of understanding of the background of the invention and it may therefore contain information that does not form the prior art that is already known to a person of ordinary skill in the art.

### SUMMARY OF INVENTION

### Technical Problem

In an aspect, it is intended to provide a composition containing a theasapogenol derivative which promotes collagen biosynthesis and thus has an excellent effect of tightening pores(or skin pores), inhibiting or improving wrinkles(or skin wrinkles), and enhancing skin elasticity as an active ingredient in the present specification.

In another aspect, it is intended to provide a composition containing a theasapogenol derivative which inhibits the 5α-reductase activity and thus has an excellent effect of controlling sebum and preventing or improving acne skin as an active ingredient in the present specification.

### Solution to Problem

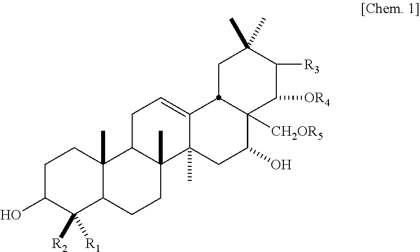

(Where R₁ and R₂ are each independently —H, C₁₋₆ alkyl, —OH, —R₆OH, or —CHO,

R₃ is —H, C₁₋₆ alkyl, —OH, or —OOCR₇,

R₄ is —H or —COR₈, and

R₅ is —H or C₁₋₆ alkyl,

where R₆ is C₁₋₆ alkyl, R₇ is C₂₋₆ alkenyl, and R₈ is C₁₋₆ alkyl).

In an aspect, the technology disclosed herein provides a composition for tightening of pores(or skin pores) containing a theasapogenol derivative represented by the chemical formula (1) as an active ingredient.

In another aspect, the technology disclosed herein provides a composition for inhibition or improvement of wrinkles(or skin wrinkles) containing a theasapogenol derivative represented by the chemical formula (1) as an active ingredient.

In still another aspect, the technology disclosed herein provides a composition for enhancement of skin elasticity containing a theasapogenol derivative represented by the chemical formula (1) as an active ingredient.

In yet another aspect, the technology disclosed herein provides a composition for control of sebum containing a theasapogenol derivative represented by the chemical formula (1) as an active ingredient.

In still yet another aspect, the technology disclosed herein provides a composition for prevention or improvement of acne skin containing a theasapogenol derivative represented by the chemical formula (1) as an active ingredient.

According to an aspect, the technology disclosed herein provides a theasapogenol derivative represented by the chemical formula (1) for use in one or more applications selected from the group consisting of tightening of pores(or skin pores), inhibition or improvement of wrinkles(or skin wrinkles), enhancement of skin elasticity, control of sebum, and prevention or improvement of acne skin.

In still another aspect, the technology disclosed herein provides one or more methods selected from the group consisting of a method of tightening pores(or skin pores), a method of inhibiting or improving wrinkles(or skin wrinkles), a method of enhancing skin elasticity, a method of controlling sebum, and a method of preventing or improving acne skin, the methods include administering a theasapogenol derivative represented by the chemical formula (1) or a composition which contains the theasapogenol derivative as an active ingredient and has one or more applications selected from the group consisting of tightening of pores(or skin pores), inhibition or improvement of wrinkles(or skin wrinkles), enhancement of skin elasticity, control of sebum, and prevention or improvement of acne skin to a subject in need thereof. In an aspect, the administration may be performed according to the administration methods and dosages described herein.

In another aspect, the technology disclosed herein provides an application of a theasapogenol derivative represented by the chemical formula (1) for preparation of a composition to be used in one or more applications selected from the group consisting of tightening of pores (or skin pores), inhibition or improvement of wrinkles (or skin wrinkles), enhancement of skin elasticity, control of sebum, and prevention or improvement of acne skin.

According to an exemplary embodiment, the theasapogenol derivative may be 21-O-angeloyltheasapogenol E3.

According to an exemplary embodiment, 21-O-angeloyltheasapogenol E3 may be represented by the following chemical formula (2).

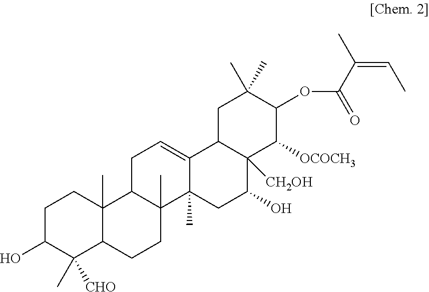

According to an exemplary embodiment, the theasapogenol derivative may be derived from green tea saponin.

According to an exemplary embodiment, the active ingredient may be contained at from 0.001 to 20% by weight based on the total weight of the composition.

According to an exemplary embodiment, the composition may promote collagen biosynthesis.

According to an exemplary embodiment, the composition may inhibit the 5α-reductase activity.

According to an exemplary embodiment, the composition may be a pharmaceutical composition.

According to an exemplary embodiment, the composition may be a cosmetic composition.

According to an exemplary embodiment, the composition may be a food composition.

### Advantageous Effects of Invention

In an aspect, the technology disclosed herein has an effect of providing a composition containing a theasapogenol derivative which promotes collagen biosynthesis and thus has an excellent effect of tightening pores(or skin pores), inhibiting or improving wrinkles(or skin wrinkles), and enhancing skin elasticity as an active ingredient.

In another aspect, the technology disclosed herein has an effect of providing a composition containing a theasapogenol derivative which inhibits the 5α-reductase activity and thus has an excellent effect of controlling sebum and preventing or improving acne skin as an active ingredient.

In still another aspect, the technology disclosed herein has an effect of providing a pharmaceutical composition, a cosmetic composition, and a food composition for tightening of pores(or skin pores), inhibition or improvement of wrinkles(or skin wrinkles), enhancement of skin elasticity, control of sebum, and prevention or improvement of acne skin, which contain a theasapogenol derivative derived from a natural substance of a plant as an active ingredient thus do not have side effects and exhibit excellent stability.

### DESCRIPTION OF EMBODIMENTS

Hereinafter, the present invention will be described in detail.

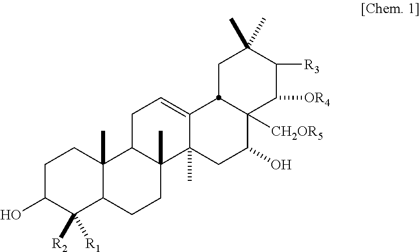

(Where R₁ and R₂ are each independently —H, C₁₋₆ alkyl, —OH, —R₆OH, or —CHO,

R₃ is —H, C₁₋₆ alkyl, —OH, or —OOCR₇,

R₄ is —H or —COR₈, and

R₅ is —H or C₁₋₆ alkyl,

where R₆ is C₁₋₆ alkyl, R₇ is C₂₋₆ alkenyl, and R₈ is C₁₋₆ alkyl).

In an aspect, the technology disclosed herein provides a composition for tightening of pores(or skin pores) containing a theasapogenol derivative represented by the chemical formula (1) as an active ingredient.

In another aspect, the technology disclosed herein provides a composition for inhibition or improvement of wrinkles(or skin wrinkles) containing a theasapogenol derivative represented by the chemical formula (1) as an active ingredient.

In still another aspect, the technology disclosed herein provides a composition for enhancement of skin elasticity containing a theasapogenol derivative represented by the chemical formula (1) as an active ingredient.

In yet another aspect, the technology disclosed herein provides a composition for control of sebum containing a theasapogenol derivative represented by the chemical formula (1) as an active ingredient.

In still yet another aspect, the technology disclosed herein provides a composition for prevention or improvement of acne skin containing a theasapogenol derivative represented by the chemical formula (1) as an active ingredient.

According to an exemplary embodiment, the theasapogenol derivative may be 21-O-angeloyltheasapogenol E3.

According to an exemplary embodiment, 21-O-angeloyltheasapogenol E3 may be represented by the following chemical formula (2). The chemical formula 2 corresponds to the theasapogenol derivative represented by the chemical formula (1) in which R₁ is —CHO, R₂ is —CH₃, R₃ is —OCOC(CH₃)═CHCH₃, R₄ is —COCH₃, and R₅ is —CH₃.

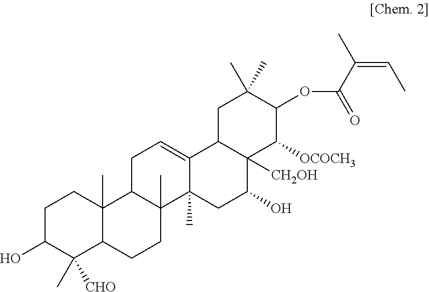

According to an exemplary embodiment, the theasapogenol derivative may be derived from green tea saponin, more specifically from green tea seed saponin. A theasapogenol derivative derived from green tea seed saponin can be manufactured by a manufacturing method including a step of obtaining an extract containing saponin from green tea seed by using water or an organic solvent; and a step of hydrolyzing the extract with an acid, a base, an enzyme, or a microorganism producing the enzyme to separate the theasapogenol derivative, more specifically, 21-O-angeloyltheasapogenol E3.

As the organic solvent, one or more organic solvents selected from the group consisting of ethanol, methanol, butanol, ether, ethyl acetate, and chloroform or any mixture of these with water may be used, and 50% ethanol may be used in an aspect.

As the acid, one or more acids selected from the group consisting of hydrochloric acid, sulfuric acid, and nitric acid or any mixed solvent of the acids with one or more alcohols selected from the group consisting of ethanol, methanol, and butanol may be used.

As the base, one or more bases selected from the group consisting of sodium hydroxide and potassium hydroxide or any mixed solvent of the bases and with one or more alcohols selected from the group consisting of ethanol, methanol, and butanol may be used.

The enzyme or the microorganism producing the enzyme is an enzyme which decomposes the sugar binding of green tea saponin contained in the extract or a microorganism producing the enzyme which decomposes the sugar binding, and it can remove the sugar moiety of green tea saponin to produce a theasapogenol derivative, more specifically, 21-O-angeloyltheasapogenol E3.

In addition, the enzyme may be one or more selected from the group consisting of glucosidase, arabinosidase, rhamnosidase, xylosidase, cellulase, hesperidinase, naringinase, glucuronidase, pectinase, galactosidase, and amyloglucosidase.

Furthermore, the microorganism producing the enzyme may be one or more selected from the group consisting of Aspergillus, Bacillus, Penicillium, Rhizopus, Rhizomucor, Talaromyces, Bifidobacterium, Mortierella, Cryptococcus, and Microbacterium,

As described above, hydrolysis is performed by using an acid, a base, an enzyme, or a microorganism producing the enzyme, and the reaction solution is concentrated under reduced pressure to remove the solvent, an alcohol is added to the residue, and the mixture is stirred from 1 to 5 times, the precipitated salts are then removed by filtration, and the filtrate is concentrated under reduced pressure to obtain a crude product, the crude product thus obtained is separated by silica gel column chromatography (chloroform:methanol=8:1 to 4:1), thereby obtaining 21-O-angeloyltheasapogenol E3.

In an aspect, the method of manufacturing theasapogenol derived from green tea saponin is disclosed in Korean Patent Application No. 10-2008-0088127, the entire contents of which are incorporated herein by reference.

According to an exemplary embodiment, the active ingredient may be contained at from 0.001 to 20% by weight based on the total weight of the composition. According to another exemplary embodiment, the active ingredient may be contained at from 0.01 to 15% by weight, from 0.01 to 10% by weight, or from 0.1 to 5% by weight based on the total weight of the composition.

In an aspect, the theasapogenol derivative represented by the chemical formula (1) or 21-O-angeloyltheasapogenol E3 to be contained in the composition disclosed herein may be contained at 0.001% by weight or more, 0.01% by weight or more, 0.1% by weight or more, or 1.0% by weight or more based on the total weight of the composition. In another aspect, the theasapogenol derivative or 21-O-angeloyltheasapogenol E3 may be contained at 20% by weight or less, 15% by weight or less, 10% by weight or less, or 5% by weight or less based on the total weight of the composition. The content is not particularly limited to the content described above, but as the content is 0.001% by weight or more, the composition exhibits an excellent effect of tightening pores(or skin pores), improving wrinkles(or skin wrinkles), enhancing skin elasticity, controlling sebum, and improving acne. It is easy to secure safety or to manufacture the composition into a formulation and excellent efficacy can be exhibited without side effects as the content is 20% by weight or less.

According to an exemplary embodiment, the composition may promote collagen biosynthesis. More specifically, the composition may increase type I procollagen synthesis of fibroblasts.

According to an exemplary embodiment, the composition may inhibit 5α-reductase activity.

According to an exemplary embodiment, the composition may be a pharmaceutical composition.

The pharmaceutical composition may additionally contain a preservative, a stabilizer, a wetting or emulsifying agent, a pharmaceutical adjuvant such as a salt and/or a buffer for the control of osmotic pressure, and other therapeutically useful substances in addition to the theasapogenol derivative represented by the chemical formula (1) or 21-O-angeloyltheasapogenol E3. The pharmaceutical composition may be formulated into various forms of oral or parenteral administration agents by conventional methods.

Examples of the oral administration agent may include tablets, pills, hard and soft capsules, liquids, suspensions, emulsions, syrups, powders, powder remedies, infinitesimal grains, granules, and pellets. These formulations may contain a surfactant, a diluent (for example, lactose, dextrose, sucrose, mannitol, sorbitol, cellulose, or glycine), and a lubricant (for example, silica, talc, stearic acid and magnesium or calcium salt thereof, and polyethylene glycol) in addition to the active ingredient. Tablets may also contain a binder such as magnesium aluminum silicate, starch paste, gelatin, tragacanth, methylcellulose, sodium carboxymethylcellulose, or polyvinylpyrrolidone. Tablets may optionally contain pharmaceutical additives such as starch, agar, a disintegrant such as alginic acid or sodium salt thereof, an absorbent, a colorant, a flavoring agent, and a sweetening agent. The tablets may be manufactured by conventional mixing, granulating, or coating methods.

In addition, the parenteral administration form may be a transdermal administration form, and for example, the parenteral administration form may be formulations such as injections, drops, ointments, lotions, gels, creams, sprays, suspensions, emulsions, suppositories, and patches, but it is not limited thereto.

The pharmaceutical composition may be administered by a parenteral form, a rectal form, a topical form, a transdermal form, a subcutaneous form, and the like. The pharmaceutical composition according to an embodiment of the present invention may be topically administered, for example, to the scalp.

The dosage of the active ingredient is determined by those skilled in the art, and the daily dose of the drug depends on various factors such as the degree of progress, time of onset, age, health condition, complications, and the like of the subject to be administered. The composition may be administered in an amount of 1 μg/kg to 200 mg/kg in an aspect and 50 μg/kg to 50 mg/kg in another aspect from one to three times a day when it is administered to an adult. The dosage is not intended to limit the scope of the present invention in any way.

The pharmaceutical composition may be an external preparation for skin, and the external preparation for skin is a generic term that may include any preparation to be applied from the outside of the skin, and various formulations of medicines may be included therein.

According to an exemplary embodiment, the composition may be a cosmetic composition.

The cosmetic composition may additionally contain functional additives and components to be contained in a general cosmetic composition in addition to the theasapogenol derivative represented by the chemical formula (1) or 21-O-angeloyltheasapogenol E3. The functional additives may include components selected from the group consisting of water-soluble vitamins, oil-soluble vitamins, polymer peptides, polymeric polysaccharides, sphingolipids, and seaweed extracts. Examples of components to be additionally blended other than these may include oil and fat components, a moisturizer, an emollient, a surfactant, organic and inorganic pigments, an organic powder, an ultraviolet absorber, a preservative, a bactericide, an antioxidant, a plant extract, a pH adjusting agent, an alcohol, a colorant, a perfume, a blood circulation accelerator, a coolant, an antiperspirant, and purified water.

The formulation of the cosmetic composition is not particularly limited and may be appropriately selected depending on the purpose. For example, the cosmetic composition may be manufactured into any one or more formulations selected from the group consisting of a skin lotion, a skin softener, a skin toner, an astringent, a lotion, a milky lotion, a moisturizing lotion, a nourishing lotion, a massage cream, a nourishing cream, a hand cream, a foundation, an essence, a nourishing essence, a pack, a soap, a cleansing foam, a cleansing lotion, a cleansing cream, a body lotion, and a body cleanser, but it is not limited thereto.

In a case in which the formulation of the present invention is a paste, a cream, or a gel, an animal fiber, a plant fiber, wax, paraffin, starch, tragacanth, a cellulose derivative, polyethylene glycol, silicone, bentonite, silica, talc, or zinc oxide may be used as a carrier component.

In a case in which the formulation of the present invention is a powder or a spray, lactose, talc, silica, aluminum hydroxide, calcium silicate, or polyamide powder may be used as a carrier component. Particularly in the case of a spray, a propellant such as chlorofluorohydrocarbons, propane/butane, or dimethyl ether may be additionally contained.

In a case in which the formulation of the present invention is a solution or an emulsion, a solvent, a dissolvent, or an emulsifier is used as a carrier component. Examples thereof may include water, ethanol, isopropanol, ethyl carbonate, ethyl acetate, benzyl alcohol, benzyl benzoate, propylene glycol, 1,3-butyl glycol oil, glycerol aliphatic esters, polyethylene glycol, or sorbitan fatty acid esters.

In a case in which the formulation of the present invention is a suspension, a liquid diluent such as water, ethanol, or propylene glycol, a suspending agent such as ethoxylated isostearyl alcohol, polyoxyethylene sorbitol ester, or polyoxyethylene sorbitan ester, microcrystalline cellulose, aluminum metahydroxide, bentonite, agar, tragacanth, or the like may be used as a carrier component.

In a case in which the formulation of the present invention is a surfactant-containing cleansing, an aliphatic alcohol sulfate, an aliphatic alcohol ether sulfate, a sulfosuccinic acid monoester, an isethionate, an imidazolinium derivative, a methyltaurate, a sarcosinate, a fatty acid amide ether sulfate, an alkylamidobetaine, an aliphatic alcohol, a fatty acid glyceride, a fatty acid diethanolamide, a vegetable oil, a linolenic derivative, or an ethoxylated glycerol fatty acid ester may be used as a carrier component.

According to an exemplary embodiment, the composition may be a food composition.

The food composition may be a formulation in a liquid or solid state. Examples thereof may include various foods, beverages, gums, tea, vitamin complexes, and health supplement foods. The food composition may be used in the form of powders, granules, tablets, capsules, or beverages. Components commonly used in the field other than the active ingredient may be appropriately selected and blended in each formulation of the food composition in addition to the active ingredient by those skilled in the art depending on the purpose of formulation or use without difficulty. A synergistic effect can be obtained when the food composition is simultaneously applied with other raw materials.

There are no particular limitations on the liquid components that can be contained in addition to the active ingredient disclosed herein, and various flavoring agents or natural carbohydrates may be contained as additional components as in ordinary beverages. Examples of the natural carbohydrate may include saccharides such as monosaccharides, disaccharides such as glucose and fructose, polysaccharides such as maltose and sucrose, dextrin, and cyclodextrin and sugar alcohols such as xylitol, sorbitol, and erythritol. As the flavoring agent, natural flavoring agents (thaumatin, stevia extract (for example, rebaudioside A or glycyrrhizin) and synthetic flavoring agents (for example, saccharin and aspartame) may be advantageously used. The ratio of the natural carbohydrate may be generally about from 1 to 20 g per 100 ml of the composition disclosed herein and about from 5 to 12 g in an aspect.

In an aspect, the food composition may contain various nutrients, vitamins, minerals (electrolytes), flavoring agents such as synthetic flavoring agents and natural flavoring agents, a colorant and an enhancer (cheese, chocolate, or the like), pectic acid and any salt thereof, alginic acid and any salt thereof, an organic acid, a protective colloid thickener, a pH adjusting agent, a stabilizer, a preservative, glycerin, an alcohol, and a carbonating agent used in carbonated beverages. In another aspect, the food composition may contain flesh for the production of natural fruit juice and vegetable beverages. These components may be used independently or in combination. The ratio of the additives may be various, but it is general that the additives are selected in the range of from 0.001 to about 20 parts by weight per 100 parts by weight of the composition disclosed herein.

### EXAMPLES

Hereinafter, the present invention will be described in more detail with reference to Examples. These Examples are for illustrating the present invention only, and it will be apparent to those skilled in the art that the scope of the present invention is not construed as being limited by these Examples.

### Preparation Example 1. Preparation of Theasapogenol Derivative

To 2 kg of green tea seed, 6 L of hexane was added, and the mixture was stirred at room temperature for degreasing. Thereafter, 4 L of 50% ethanol was added to 1 kg of the degreased green tea seed, the mixture was refluxed three times for extraction and then immersed at 15° C. for 1 day. Thereafter, the residue and the filtrate were separated by filtration through a filter cloth and centrifugation, and the separated filtrate was concentrated under reduced pressure, the extract thus obtained was suspended in water and then extracted five times with 1 L of ether to remove the pigment, and the aqueous layer was extracted three times with 500 ml of 1-butanol. The entire 1-butanol layer thus obtained was concentrated under reduced pressure to obtain a 1-butanol extract, this was dissolved in a small amount of methanol, the solution was then added to a large amount of ethyl acetate, and the precipitate thus formed was dried, thereby obtaining 300 g of green tea seed extract.

To 10 g of the green tea seed extract thus obtained, IN HCl-50% methanol solution (v/v) was added to be 20-fold (v/w) the extract, and the mixture was heated and refluxed in a water bath at 80° C. for 8 hours to hydrolyze the saccharides bound to green tea seed saponin. The reaction solution was concentrated under reduced pressure to remove the solvent. Ethanol (200 ml) was added to the residue and the mixture was stirred (3 times), and the precipitated salt was removed by filtration, and the filtrate was concentrated under reduced pressure to obtain a crude product. Thereafter, the crude product thus obtained was separated by silica gel column chromatography (chloroform:methanol=7:1 to 3:1), thereby obtaining 0.55 g of 21-O-angeloyltheasapogenol E3 of a theasapogenol derivative.

It has been confirmed that the product is 21-O-angeloyltheasapogenol E3 by using the Varian Gemini 2000 300 MHz (Varian). As a result, the same result as in Experimental Example 1 of the detailed description of Patent Application No. 10-2008-0088127 referred to herein has been obtained.

### Test Example 1. Promotion of Collagen Biosynthesis

The collagen biosynthesis promoting effect of the theasapogenol derivative obtained in Preparation Example 1 was measured in comparison with TGF-beta of a positive control.

Fibroblasts were seeded by 10⁵ in one well of a 24-well plate and incubated until they grew up to about 90%. This was incubated in serum-free DMEM medium for 24 hours and treated with 2 μg/ml of each of the theasapogenol derivative obtained in Preparation Example 1 and TGF-beta, and incubated in a CO₂ incubator for 24 hours. Thereafter, the supernatant was taken out and subjected to the observation using the ELISA kit (procollagen type (I)) to determine whether the procollagen increased or decreased. The results are presented in Table 1, and the collagen synthesizing ability was compared by setting the collagen synthesizing ability of the untreated group to 100.

TABLE 1

| Division | Collagen synthesizing ability (%) |
|---|---|
| Untreated group | 100 |
| TNF-beta | 183.5 ± 13.1 |
| Preparation Example 1 | 145.1 ± 12.5 |

From the results presented in Table 1 above, it has been confirmed that the theasapogenol derivative disclosed herein exhibits high collagen synthesizing ability. Consequently, it can be seen that the theasapogenol derivative disclosed herein increases the amount of collagen produced around the pores(or skin pores) and thus has an effect of tightening the enlarged skin pores, inhibiting or improving skin wrinkles, and enhancing skin elasticity.

### Test Example 2. Inhibition of 5α-Reductase Activity

The 5α-reductase activity inhibiting effect was measured by the ratio of [¹⁴C]testosterone converted into [¹⁴C]dihydrotestosterone in HEK293-5αR2 cells. HEK293 cells were transfected with p3×FLAG-CMV-5αR2 and incubated in a 24-well plate at a density of 2.5×10⁵ cells per well (Park et al., 2003, JDS Vol. 31, pp. 91-98). The next day, the medium was replaced with a fresh medium containing an enzyme substrate and an inhibitor, and 0.05 μCi [¹⁴C]testosterone (Amersham Pharmacia biotech, UK) was used as the substrate of the medium. In order to confirm the degree of inhibition, 2 μg/ml of theasapogenol derivative obtained in Preparation Example 1 was added to the medium as a test substance, and the medium was incubated in a 5% CO₂ incubator at 37° C. for 2 hours. For comparison, a medium which did not contain any of the test substances was used as a negative control group and one prepared by adding 2 μg/ml of Finasteride to a medium and incubating the medium under the same conditions was used as a positive control group.

Thereafter, the incubated mediums were collected and testosterone was extracted with 800 μl of ethyl acetate. The upper organic solvent layer was separated and dried, and the residue was dissolved again in 50 μl of ethyl acetate and developed on the silica plastic sheet kieselgel 60 F254 by using ethyl acetate-hexane (1:1) as a developing solvent. After the plastic sample was dried in the air, a bath system was used to measure the amounts of isotopes, the dried plastic sheet and an x-ray film were placed in a bath cassette together, and the amounts of isotopes in testosterone and dihydrotestosterone remaining on the film after one week were measured. The results are presented in the following Table 2.

TABLE 2

| Sample | Conversion rate (%) | Inhibition rate (%) |
|---|---|---|
| Preparation Example 1 | 30 | 38 |
| Control group | 48 | — |
| Positive control (Finasteride) | 27 | 44 |

(1) Conversion rate: radioactivity to DHT area/total radioactivity

(2) Inhibition rate: 100×(conversion rate of control group−conversion rate of sample)/conversion rate of control group

It can be seen that the theasapogenol derivative disclosed herein exhibits superior 5α-reductase activity inhibiting efficacy to the control group as a result of effectiveness evaluation, and thus has an excellent effect of controlling sebum, and preventing or improving acne skin.

From the results presented in Table 2 above, it can be seen that the theasapogenol derivative blocks the conversion of testosterone into dihydrotestosterone by effectively inhibiting the activity of 5α-reductase which converts testosterone into dihydrotestosterone, binds to the receptor protein in the cytoplasm, enters the nucleus, activates sebaceous gland cells, promotes differentiation, and causes hypersecretion of sebum in the sebaceous glands. Consequently, the theasapogenol derivative disclosed herein has an effect of inhibiting 5α-reductase activity, inhibiting hypersecretion of sebum, controlling sebum, and preventing or improving acne.

### Test Example 3. Skin Pore Tightening

Example and Comparative Example of a lotion formulation (unit: % by weight) were prepared as follows by using the theasapogenol derivative obtained in Preparation Example 1.

TABLE 3

| | | Comparative |
|---|---|---|
| Name of raw material | Example | Example |
| 1. Cetearyl alcohol | 1.0 | 1.0 |
| 2. Glyceryl stearate, lipophilic | 1.0 | 1.0 |
| 3. Glyceryl stearate SE | 1.5 | 1.5 |
| 4. Phyto squalane | 3 | 3 |
| 5. Hydrogenated polydecene | 2 | 2 |
| 6. Dimethicone | 0.5 | 0.5 |
| 7. Polysorbate 60 | 1 | 1 |
| 8. Sorbitan sesquioleate | 0.4 | 0.4 |
| 9. Methylparaben | 0.1 | 0.1 |
| 10. Propylparaben | 0.05 | 0.05 |
| 11. Purified water | To 100 | To 100 |
| 12. Butylene glycol | 5 | 5 |
| 13. Polyacrylate-13* Polyisobutene* | 0.5 | 0.5 |
| Polysorbate 20 | | |
| 14. Preparation Example 1 | 1 | 0 |

1) The components 11 to 14 were uniformly mixed while heating to 70° C. to prepare an aqueous phase part.

2) The components 1 to 10 were uniformly mixed while heating to 70° C. to prepare an oil phase part.

3) The oil phase part of 2) was put into the aqueous phase part of 1) and homomixed at 7,200 rpm for 6 minutes.

4) The mixture of 3) was cooled to room temperature.

The lotion formulations of Example and Comparative Example thus prepared were applied onto the face of 10 subjects of male and female having a wide pore size every day for 4 weeks. The judgment on the pore tightening effect was performed by taking photographs of the face before the experiment and after 4 weeks from the start of the experiment and by visually evaluating the difference by the experts. The results are presented in Table 4 (evaluation criteria: 0 (pores have not been tightened at all) to 5 (pores have been significantly tightened)).

TABLE 4

| Substance | Evaluated grade |
|---|---|
| Example | 3.2 |
| Comparative Example | 0.8 |

In the case of Example containing the theasapogenol derivative obtained in Preparation Example 1, the pore tightening effect was obtained. However, in the case of Comparative Example not containing the theasapogenol derivative, there was no pore tightening effect as the evaluated grade was 0.8. From the above results, it has been found that the theasapogenol derivative disclosed herein has an excellent pore tightening effect.

Formulation examples of the composition according to an aspect of the present invention are described below, but the composition may be applied to various other formulations, and the formulation examples are not intended to limit the present invention but only to illustrate the present invention.

### [Formulation Example 1] Shampoo

Shampoo was prepared by a conventional method so as to have the composition presented in the following Table 5.

TABLE 5

| Component | Content (% by weight) |
|---|---|
| Theasapogenol derivative represented | 2.00 |
| by chemical formula (1) or | |
| 21-O-angeloyltheasapogenol E3 | |
| Sodium lauryl sulfate | 10.00 |
| Cocamidopropyl betaine | 3.00 |
| Carboxyl vinyl polymer | 0.30 |
| Polyquaternium-10 | 0.20 |
| Cetyltrimethylammonium chloride | 0.10 |
| Purified water | Balance |
| Sum | 100.00 |

### [Formulation Example 2] Rinse

Rinse was prepared by a conventional method so as to have the composition presented in the following Table 6.

TABLE 6

| Component | Content (% by weight) |
|---|---|
| Theasapogenol derivative represented | 2.00 |
| by chemical formula (1) or | |
| 21-O-angeloyltheasapogenol E3 | |
| Cetyl alcohol | 2.00 |
| Stearyl alcohol | 2.50 |
| Behenyl alcohol | 0.50 |
| Silicone emulsion | 0.40 |
| Cyclomethicone | 1.00 |
| Dimethyldistearylammonium chloride | 0.10 |
| Purified water | Balance |
| Sum | 100.00 |

### [Formulation Example 3] Ointment

Ointment was prepared by a conventional method so as to have the composition presented in the following Table 7.

TABLE 7

| Component | Content (% by weight) |
|---|---|
| Theasapogenol derivative represented | 2.00 |
| by chemical formula (1) or | |
| 21-O-angeloyltheasapogenol E3 | |
| Glycerin | 8.00 |
| Butylene glycol | 4.00 |
| Liquid paraffin | 15.00 |
| β-Glucan | 7.00 |
| Carbomer | 0.10 |
| Caprylic/Capric triglyceride | 3.00 |
| Squalane | 1.00 |
| Cetearyl glucoside | 1.50 |
| Sorbitan stearate | 0.40 |
| Cetearyl alcohol | 1.00 |
| Beeswax | 4.00 |
| Purified water | Balance |
| Sum | 100.00 |

### [Formulation Example 4] Hair Tonic

A hair tonic was prepared by a conventional method so as to have the composition presented in the following Table 8.

TABLE 8

| Component | Content (% by weight) |
|---|---|
| Ethanol | 55.0 |
| Castor oil | 5.00 |
| Glycerin | 3.00 |
| Piroctone olamine | 0.10 |
| Theasapogenol derivative represented | 1.00 |
| by chemical formula (1) or | |
| 21-O-angeloyltheasapogenol E3 | |
| Perfume and colorant | Proper amounts |
| Purified water | Balance |
| Sum | 100.00 |

### [Formulation Example 5] Hair Lotion

Hair lotion was prepared by a conventional method so as to have the composition presented in the following Table 9.

TABLE 9

| Component | Content (% by weight) |
|---|---|
| Cetostearyl alcohol | 2.00 |
| Stearyltriethylammonium chloride | 2.00 |
| Hydroxyethylcellulose | 0.50 |
| Piroctone olamine | 0.10 |
| Theasapogenol derivative represented | 10.0 |
| by chemical formula (1) or | |
| 21-O-angeloyltheasapogenol E3 | |
| Perfume and colorant | 0.50 |
| Purified water | Balance |
| Sum | 100.00 |

### [Formulation Example 6] Soap

Soap was prepared by a conventional method so as to have the composition presented in the following Table 10.

TABLE 10

| Component | Content (% by weight) |
|---|---|
| Theasapogenol derivative represented | 5.00 |
| by chemical formula (1) or | |
| 21-O-angeloyltheasapogenol E3 | |
| Titanium dioxide | 0.20 |
| Polyethylene glycol | 0.80 |
| Glycerin | 0.50 |
| Ethylenediamine tetraacetic acid | 0.05 |
| Sodium | 1.00 |
| Colorant | Proper amount |
| Soap incense | Proper amount |
| Soap base (moisture content: 13%, | to 100 |
| parts by weight) | |
| Sum | 100.00 |

### [Formulation Example 7] Lotion

Lotion was prepared by a conventional method so as to have the composition presented in the following Table 11.

TABLE 11

| Component | Content (% by weight) |
|---|---|
| Theasapogenol derivative represented | 2.00 |
| by chemical formula (1) or | |
| 21-O-angeloyltheasapogenol E3 | |
| L-ascorbic acid-2-phosphate | 1.00 |
| magnesium salt | |
| Water-soluble collagen (1% aqueous | 1.00 |
| solution) | |
| Sodium citrate | 0.10 |
| Citric acid | 0.05 |
| Licorice extract | 0.20 |
| 1,3-Butylene glycol | 3.00 |
| Purified water | Balance |
| Sum | 100.00 |

### [Formulation Example 8] Cream

Cream was prepared by a conventional method so as to have the composition presented in the following Table 12.

TABLE 12

| Component | Content (% by weight) |
|---|---|
| Theasapogenol derivative represented | 2.00 |
| by chemical formula (1) or | |
| 21-O-angeloyltheasapogenol E3 | |
| Polyethylene glycol monostearate | 2.00 |
| Self-emulsifying monostearate glycerin | 5.00 |
| Cetyl alcohol | 4.00 |
| Squalene | 6.00 |
| Tri-2-ethylhexane glyceryl | 6.00 |
| Sphingoglycolipids | 1.00 |
| 1-3-Butylene glycol | 7.00 |
| Purified water | Balance |
| Sum | 100.00 |

### [Formulation Example 9] Pack

Pack was prepared by a conventional method so as to have the composition presented in the following Table 13.

TABLE 13

| Component | Content (% by weight) |
|---|---|
| Theasapogenol derivative represented | 2.00 |
| by chemical formula (1) or | |
| 21-O-angeloyltheasapogenol E3 | |
| Polyvinyl alcohol | 13.00 |
| L-ascorbic acid-2-phosphate | 1.00 |
| magnesium salt | |
| Lauroylhydroxyproline | 1.00 |
| Water-soluble collagen (1% aqueous | 2.00 |
| solution) | |
| 1,3-Butylene glycol | 3.00 |
| Ethanol | 5.00 |
| Purified water | Balance |
| Sum | 100.00 |

### [Formulation Example 10] Cosmetic Liquid Preparation

A cosmetic liquid preparation was prepared by a conventional method so as to have the composition presented in the following Table 14.

TABLE 14

| Component | Content (% by weight) |
|---|---|
| Theasapogenol derivative represented | 2.00 |
| by chemical formula (1) or | |
| 21-O-angeloyltheasapogenol E3 | |
| Hydroxyethylene cellulose (2% | 12.00 |
| aqueous solution) | |
| Xanthan gum (2% aqueous solution) | 2.00 |
| 1,3-Butylene glycol | 6.00 |
| Concentrated glycerin | 4.00 |
| Sodium hyaluronate (1% aqueous | 5.00 |
| solution) | |
| Purified water | Balance |
| Sum | 100.00 |

### [Formulation Example 11] Soft Capsule

With 50 mg of the theasapogenol derivative represented by the chemical formula (1) or 21-O-angeloyltheasapogenol E3, 80 to 140 mg of L-carnitine, 180 mg of soybean oil, 2 mg of palm oil, 8 mg of hydrogenated vegetable oil, 4 mg of yellow wax, and 6 mg of lecithin were mixed, and the mixture was filled in a capsule by 400 mg per one capsule by a conventional method, thereby preparing a soft capsule.

### [Formulation Example 12] Tablet

With 50 mg of the theasapogenol derivative represented by the chemical formula (1) or 21-O-angeloyltheasapogenol E3, 200 mg of galactooligosaccharide, 60 mg of lactose, and 140 mg of maltose were mixed, the mixture was granulated by using a fluidized bed drier, 6 mg of sugar ester was added to the granule, and the mixture was formed into a tablet by using a tableting machine.

### [Formulation Example 13] Granule

With 50 mg of the theasapogenol derivative represented by the chemical formula (1) or 21-O-angeloyltheasapogenol E3, 250 mg of anhydrous crystalline glucose and 550 mg of starch were mixed, the mixture was formed into granules by using a fluidized bed granulator, and the granules were filled in a pouch, thereby preparing granules.

### [Formulation Example 14] Health Drink

With 50 mg of the theasapogenol derivative represented by the chemical formula (1) or 21-O-angeloyltheasapogenol E3, 10 g of glucose, 0.6 g of citric acid, and 25 g of liquid oligosaccharide were mixed, 300 ml of purified water was added to the mixture, and the mixture thus obtained was filled in a bottle by 200 ml per one bottle. Thereafter, the bottled drink was sterilized at 130° C. for 4 to 5 seconds, thereby preparing a health drink beverage.

While the present invention has been described with respect to the specific embodiments, it will be apparent to those skilled in the art that the above embodiments are not limiting but illustrative and that the scope of the present invention is not limited thereby. Therefore, it should be understood that the actual scope of the present invention will be defined by the appended claims and their equivalents.

1. A method for tightening of skin pores, control of sebum, or prevention or improvement of acne skin comprising administering an effective amount of a theasapogenol derivative represented by the following chemical formula (1) to a subject in need thereof: 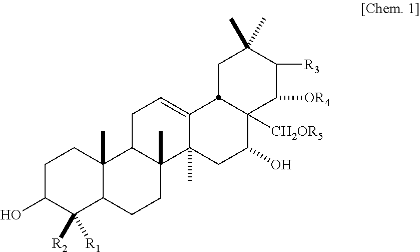
wherein R₁ and R₂ are each independently —H, C₁₋₆ alkyl, —OH, —R₆OH, or —CHO,
R₃ is —H, C₁₋₆ alkyl, —OH, or —OOCR₇,
R₄ is —H or —COR₈, and
R₅ is —H or C₁₋₆ alkyl,
wherein R₆ is C₁₋₆ alkyl, R₇ is C₂₋₆ alkenyl, and R₅ is C₁₋₆ alkyl.

2. A method for inhibition or improvement of skin wrinkles, or enhancement of skin elasticity comprising a theasapogenol derivative represented by the following chemical formula (1) as an active ingredient: 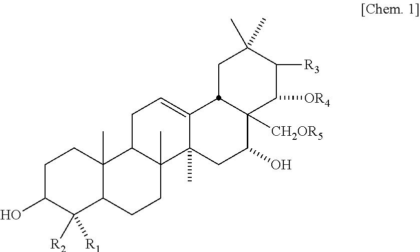
wherein R₁ and R₂ are each independently —H, C₁₋₆ alkyl, —OH, —R₆OH, or —CHO,
R₃ is —H, C₁₋₆ alkyl, —OH, or —OOCR₇,
R₄ is —H or —COR₈, and
R₅ is —H or C₁₋₆ alkyl,
wherein R₆ is C₁₋₆ alkyl, R₇ is C₂₋₆ alkenyl, and R₅ is C₁₋₆ alkyl.

3-5. (canceled)

6. The method according to claim 1, wherein the theasapogenol derivative is 21-O-angeloyltheasapogenol E3.

7. The method according to claim 6, wherein 21-O-angeloyltheasapogenol E3 is represented by the following chemical formula (2):

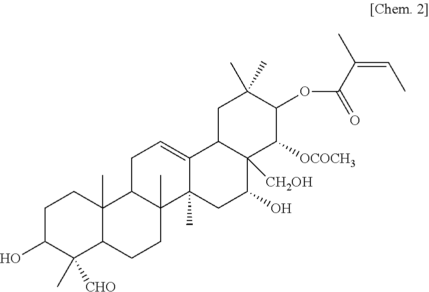

8. The method according to claim 1, wherein the theasapogenol derivative is derived from green tea saponin.

9. The method according to claim 1, wherein the active ingredient is contained at from 0.001 to 20% by weight based on a total weight of the composition.

10. The method according to claim 1, wherein the composition promotes collagen biosynthesis.

11. The method according to claim 1, wherein the composition inhibits 5α-reductase activity.

12. The method according to claim 1, wherein the composition is a pharmaceutical composition, a cosmetic composition, or a food composition.

13. (canceled)

14. (canceled)
